(19) United States
(12) Patent Application Publication
(10) Pub. No.: US 2010/0298911 A1
(43) Pub. Date: Nov. 25, 2010

(54) Personalized interactive laser therapy in real time

(76) Inventor: Gal Shafirstein, Little Rock, AR (US)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 12/661,014

(22) Filed: Mar. 9, 2010

Related U.S. Application Data
(60) Provisional application No. 61/209,578, filed on Mar. 9, 2009.

(51) Int. Cl. A61N 5/067

(52) U.S. Cl. 607/89

(57) ABSTRACT

A method and apparatus for personalized interactive laser therapy (PILT) treatment of Port Wine Stains (PWS) in real time in which low radiant exposure from a laser hand piece slightly heats up the treatment site while an infrared sensor in the laser hand piece measures the temperature increase. A mathematical algorithm calculates the vessel size distribution at the treatment site. The appropriate radiant exposure (based on the vessel size distribution) is then delivered to the treatment site.

### CROSS-REFERENCE TO RELATED APPLICATIONS

This application claims the benefit of U.S. provisional application 61/209,578, filed on Mar. 9, 2009, the disclosure of which is incorporated herein by reference in its entirety.

### STATEMENT REGARDING FEDERALLY SPONSORED RESEARCH OR DEVELOPMENT

Not applicable

### BACKGROUND OF THE INVENTION

1. Field of the Invention

The present invention relates to personalized interactive laser treatment in real time, and in particular, to a method and apparatus for personalized interactive laser therapy (PILT) in real time to improve the response of superficial tumors, hair removal, Port Wine Stain (PWS) and the like to laser treatment by customizing the laser treatment for specific conditions.

2. Brief Description of the Related Art

Port-wine stains (PWS) are birthmarks that affect 850,000 to 1,500,000 children in the US and cause significant psychosocial ramifications that worsen with age.[1-6] Although any area of the body may be involved, about 90% of PWS occur on the face or neck.[7] As the topographic distribution appears to be segmental, the surface area involved is usually extensive, with patients typically having one or more dermal regions involved.[8,9] The presence of a PWS can have a devastating impact on a child's psychosocial development, and low self-esteem and introversion are common personality traits among children with PWS.[1.2.10,11] These birthmarks do not resolve and typically worsen with age.[6,12,13] PWS always present at birth. Over time, these blood vessels become progressively more ectatic (that is, dilated or distended), and the involved tissue becomes thickened and nodular with the maturation of the individual.[8] Although slow, this gradual change in size of the vessels leads to deepening in the color of the birthmark and changes in the texture of the skin.[14] The pale pink, flat PWS birthmarks observed in babies become dark red or purple and nodular with maturity.[12] Nodular lesions may bleed spontaneously or in response to minor trauma. Some PWS lesions are associated with hypertrophy of underlying and adjacent tissues, producing asymmetry of the affected side.

If the PWS is not treated or does not respond to laser treatment, progressive hypertrophy changes not only distort anatomical structures lying within the affected area, but could also alter functionality, especially when the face and limbs are affected.[6,13] In some cases, complex surgical interventions are required to treat the lesion.[15]

The range of PWS blood vessels that need to be coagulated has limited the efficacy of current laser treatment and requires the use of multiple lasers to achieve satisfactory results.[16,17] To date, only 10% of PWS are completely cleared using standard laser therapy and the treated PWS worsen in 35% of the cases.[15,17-19]

PWS are malformations of dermal venulocapillary vessels.[15] The number of vessels does not increase over time, but they become more ectatic.[15,23] One hypothesis is that the progressive ectasia of vessels that occurs with age is due to a congenital defect in the maintenance of vascular tone.[15,23] A decrease in density of nerve twigs around these abnormally ectatic blood vessels has been documented by various immunohistochemical studies.[23] Histologically, the abnormally dilated vessels are lined by flat, mitotically inactive endothelial cells and peripherally rearranged pericytes surrounded by basement membrane material.[15] Mast cells are present in normal numbers, and no anomalies in basement membrane or extracellular matrix components have been observed.[15,24] Dermal vessel diameters ranging from 20 μm to 500 μm have been reported to be scattered throughout the skin from 0.2 to 3.5 mm deep.[9,25,26] The variations in vascular morphology strengthen the need to introduce PILT in the clinical setting.

The well-known theory of selective photothermolysis proposed in 1983 by Anderson and Parrish is the premise for laser treatment of vascular disorders, such as PWS.[27,28] At selected laser wavelengths the laser energy absorbed by the oxy- and deoxyhemoglobin in blood causes coagulation of blood cells and, by heat conduction, transmural coagulation of vessel walls (i.e., photocoagulation).[27,29-31] Photocoagulation causes purpura to appear in the treated region during treatment. The purpura is a clinical indication that the treatment was successful and it resolves within few weeks.[32-35] However, the presence of purpura increases the anxiety of children to multiple laser treatments and complete clearance is only obtained in 10% of cases, and in 65% only partial clearance is achieved, whereas an additional 15-20% do not respond to flashlamp pulsed dye laser (FPDL).[15,17-19] The poor clinical response may be owing to the wide range of blood vessel size and morphology that are not taken into account in the current planning of a laser treatment.

There are no animal models that imitate or mimic PWS lesions. Hence, the laser parameters for selective photothermolysis must be determined largely through mathematical modeling. Various mathematical models have been developed to predict optimal laser parameters to achieve high-efficacy laser treatment of PWS.[20,25,32,36-40] In all of these models, the skin is considered an optically turbid medium. Light propagation in a turbid medium, such as human tissue, can be modeled through an analytical solution of diffusion theory and Monte Carlo simulations.[39,41] Shafirstein et al. developed and presented a mathematical model that is a new approach to the diffusion approximation.[20] This model predicts realistic temperature distribution within blood vessels in the dermis by including latent heat of evaporation while calculating the photon flux and thermal field simultaneously via finite element method.[20] Using the model the maximum temperature may be calculated as a function of vessel size for various laser parameters as exemplified by FIGS. 1A and 1B.

The model predictions have been validated in animal studies and used to explain clinical outcomes of FPDL and Nd:YAG treatments of PWS.[16,20,22,42-44] Using these data and further calculations, it has recently been proposed to use multiple combinations of laser parameters to treat patients with PWS as shown in Table 1.[16]

TABLE 1
Recommended laser parameters for use
in various vascular disorders.[16]

| Vessel | Vascular | |
|---|---|---|
| size (μm) | Malformation | Laser |
| 50-150 | PWS (early stage) | FPDL 585 nm, 6 J/cm², 0.45 ms, 1 P |
| | Hemangioma (*) | FPDL 595 nm, 8 J/cm², 0.45 ms, 1 P |
| 150-500 | PWS (developed) | FPDL 585 nm, 8 J/cm², 0.45 ms, 1 P |
| | Teleangiectases | FPDL 595 nm, 8 J/cm², 0.4 ms, 1 P |
| | Spider angioma | FPDL 595 nm, >12 J/cm², 0.45 ms, 3 P |
| | Venous | Nd: YAG, 100 J/cm², 10-100 ms |
| | malformation | |
| | Cherry angioma | 2.5 mm spot size |
| 500-1000 | Cherry angioma | Nd: YAG, 100-200 J/cm², 30-100 ms, |
| | Venous | 2.5 mm spot size |
| | malformation | |

(*) small vessels (<50 μm) hemangioma will have very poor response to any of these lasers

The limitations of the prior art are overcome by the present invention as described below.

### BRIEF SUMMARY OF THE INVENTION

The present invention is a method and apparatus for personalized interactive laser treatment in real time, and in particular, to a method and apparatus for personalized interactive laser therapy (PILT) in real time to improve the response of superficial tumors, hair removal, Port Wine Stain (PWS) and the like to laser treatment by customizing the laser treatment for specific conditions. While the present invention is described with specific reference to the treatment of PWS, the method and apparatus of the present invention is not so limited and may be employed in the treatment of other conditions such as, but not limited to, superficial tumors and hair removal.

There are literally hundreds of possible clinical laser settings and vessel size combinations to choose from for an effective laser treatment.[16,20-22] PILT may be accomplished by coupling real time thermal imaging of PWS with a validated mathematical model.[20-26]

However, even state of the art imaging modalities cannot be used to image vessel size distribution in PWS. Instead of direct imaging of the PWS's vasculature the present invention measures the temperature increase of PWS during laser treatment. The measured temperature is compared to the calculated temperature to estimate the vessel size distribution of the PWS and then to select the optimal laser parameters for treatment.

The present invention is an improvement over the prior art in that a real time procedure measures the vessel size distribution as follows:

Step 1. Low radiant exposure (e.g. 4-5 J/cm² for PDL or 20 J/cm² for NIR lasers) will be used to slightly heat up the treated area, during that time an infrared sensor in the laser hand piece will measure the temperature increase and a mathematical algorithm will be used to calculate the vessel size distribution at the site that will be treated. As used herein, “low” or “low level” “radiation” or “radiant exposure” means an energy intensity delivered to a treatment site that is sufficient to increase the temperature of the treatment site but less than the energy intensity required to provide any substantial permanent therapeutic effect to the treatment site. The mathematical algorithm may be any algorithm, including those known in the prior art, that has the capability of providing a calculation of vessel size distribution in the treatment area from a known temperature rise and/or rate of change of temperature, such as the thermal relaxation or rate of cooling, from a given exposure of the treatment site to low level radiation.

Step 2. Immediately after Step 1 (within milliseconds), the appropriate radiant exposure (based on the vessel size distribution) will be delivered to the treated region. A radiant exposure is considered “appropriate” if the amount of radiant exposure provides a therapeutic effect to the treatment site.

The present invention provides real time control of laser therapy. It is simple and does not require a thermal camera, since the treatment laser itself may used to determine the absorption of the vessels by the high power laser.

The pre-heating is also expected to improve the response to the therapy, since dilated vessels respond better to laser therapy.

The procedure can be applied to other types of laser therapy such as hair removal.

These and other features, objects and advantages of the present invention will become better understood from a consideration of the following detailed description of the preferred embodiments and appended claim in conjunction with the drawings as described following:

### BRIEF DESCRIPTION OF THE SEVERAL VIEWS OF THE DRAWINGS

FIGS. 1A and 1B show the maximal temperature at the center of a 1.2-mm-deep dilated vessel 0.8 mm away from the beam's center which was calculated for FPDL at different energy densities (8 J/cm² and 12 J/cm²) and pulse durations (0.45 ms and 1.5 ms) for wavelengths of 585 nm (a) and 595 nm (b).[20]

### DETAILED DESCRIPTION OF THE INVENTION

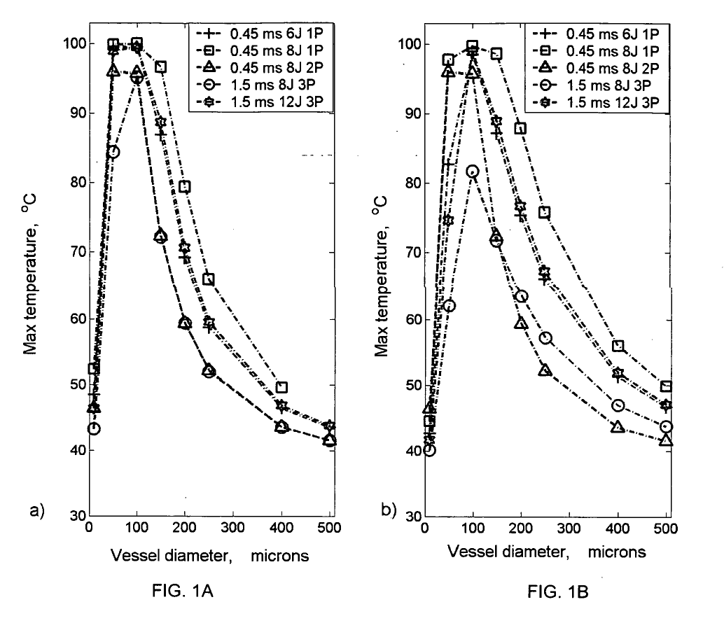

With reference to FIGS. 1A and 1B, the preferred embodiments of the present invention may be described using the treatment of PWS as an example.

Instead of direct imaging of the PWS's vasculature, the present invention measures the temperature increase of PWS during laser treatment. The measured change in temperature (dT) is compared to the calculated temperature (as exemplified by FIGS. 1A and 1B) to estimate the vessel size distribution of the PWS and then to select the optimal laser parameters for treatment. The measured rate of change of temperature (dT/dt) may also be used to refine the estimate of the vessel size distribution. More specifically, the thermal relaxation; i.e., the rate of cooling, may also be useful in performing the vessel size estimation.

The present invention is an improvement over the prior art in that a real time procedure measures the vessel size distribution. In one embodiment of the present invention the following steps would be performed:

Step 1. Use low radiant exposure (e.g. 5-8 J/cm² for PDL or 20-40 J/cm² for NIR lasers) to slightly heat up the treatment site while an infrared sensor in the laser hand piece measures the temperature increase. The infrared sensor may be operatively coupled to one or more fiber optics placed to receive infrared radiation from the treatment site. Note that a thermal camera could perform this function, but at much greater cost than a simple infrared sensor.

Step 2. Evaluate the vessel size distribution at the treatment site by using a mathematical algorithm to calculate the vessel size distribution. This evaluation is done in real time with software used to provide the laser settings for the particular treatment site. Instead of performing an actual calculation each time, an alternative is to perform a set of calculations for the various possible laser parameters, which could total 20,000 combinations, and put this information into a lookup table.

Step 3. Immediately after Step 2, the appropriate radiant exposure (based on the calculated vessel size distribution) is delivered to the treatment site. The treatment may be delivered by the same laser as used to provide the low radiant exposure in Step 1 or may be by a separate laser. Both the measurement and treatment steps will be done in real time so that the clinical response, i.e., temperature increase can be evaluated and retreatment can be done as needed.

The entire cycle should take around 10 seconds.

The mathematical algorithm may be derived from the approach described in Ref. [20], in which diffusion approximation with the finite element method was used to determine the optimal wavelength and radiant exposure to treat PWS. Alternatively, the Monte Carlo method may be utilized. The light and heat diffusion equations were simultaneously solved with the finite element method. The latent heat of evaporation was included in the thermal analysis. The temperature and coagulations patterns were calculated for various laser parameters. As exemplified by FIGS. 1A and 1B, the maximal temperature rise associated with various vessel diameters and laser treatment parameters can be determined. Therefore, by measuring the temperature rise associated with known laser parameters, the vessel size distribution can be determined. Then, from the vessel size distribution, the laser parameters necessary to achieve the desired coagulation of the blood vessels can be determined.

To date, clinicians use a trial-and-error approach to find the laser energy density (J/cm²) and pulse time that will yield the best clinical response. However, only 10% of PWS are completely cleared and in 35% the current treatment does not stop the progression of PWS, as revealed in a recent 10-year follow-up prospective clinical study.[18]

The development of personalized interactive laser therapy (PILT) for patients with PWS is expected to significantly improve the clearance rates of PWS. In addition, it is expected to reduce the number of treatments required for complete clearance of PWS. This achievement will have a huge impact on the pediatric population where these treatments must be performed under general anesthesia and the risks of multiple anesthesias are considerable. It will also have major impact on adolescents with PWS, where the psychosocial ramifications of PWS are significant and the natural progression of the disease can lead to devastating outcomes requiring multiple surgical interventions.

PILT will assist physicians to better utilize current laser systems to improve clinical outcomes of laser therapy for patients with PWS, and it could be applied (in the future) for laser treatment of other cutaneous lesions such as superficial venous and arteriovenous malformations.

The PILT could improve the chance for complete clearance of the PWS in fewer treatments, thus reducing the overall emotional stress from laser therapy and reduce number of visits to the hospital. In young children this treatment is done under general anesthesia, and the reduction of anesthesia sessions reduces the chance of complications.

The present invention may use the same laser to both evaluate and treat the selected treatment site. This has the advantage that the same optical properties apply to both the evaluation and the treatment steps. The present invention may be added to existing laser systems, including multi-laser systems.

There are laser systems that can be calibrated for a range of settings, but some clinical laser systems must be calibrated every time the radiant exposure is changed. Therefore, using the same laser to provide the low radiant exposure and the treatment could require recalibration. Possible solutions include:

(1) Adding a removable diffuser between the laser beam and the tissue. The diffuser can be controlled such that it can be removed from the beam path within a fraction of a second.

(2) Adding another beam to the same port from which the aiming beam is being delivered.

The apparatus to be added to an existing system could include an infrared sensor, associated fiber optics, spacer, optical diffuser or filter, and electronics to read the sensor, determine the laser parameters by calculating the algorithm or performing a table lookup, operating the laser to carry out the treatment, and cycling the process.

The present invention has been described with reference to certain preferred and alternative embodiments that are intended to be exemplary only and not limiting to the full scope of the present invention.

### REFERENCES

[1]. Miller A C, Pit-Ten Cate I M, Watson H S, Geronemus R G. Stress and family satisfaction in parents of children with facial port-wine stains. Pediatric Dermatology. 1999; 16:190-197.
[2]. Strauss R P, Resnick S D. Pulsed dye laser therapy for port-wine stains in children: psychosocial and ethical issues. [see comments]. Journal of Pediatrics. 1993; 122:505-510.
[3]. Hansen K, Kreiter C D, Rosenbaum M, Whitaker D C, Arpey C J. Long-term psychological impact and perceived efficacy of pulsed-dye laser therapy for patients with port-wine stains. Dermatol Surg. 2003; 29:49-55.
[4]. Augustin M, Zschocke 1, Wiek K, Peschen M, Vanscheidt W. Psychosocial stress of patients with port wine stains and expectations of dye laser treatment. Dermatology. 1998; 197:353-360.
[5]. Jacobs A H, Walton R G. The incidence of birthmarks in the neonate. Pediatrics. 1976; 58:218-222.
[6]. Klapman M H, Yao J F. Thickening and nodules in port-wine stains. Journal of the American Academy of Dermatology. 2001; 44:300-302.
[7]. Mills C M, Lanigan S W, Hughes J, Anstey A V. Demographic study of port wine stain patients attending a laser clinic: family history, prevalence of naevus anaemicus and results of prior treatment. Clinical & Experimental Dermatology. 1997; 22:166-168.
[8]. Finley J L, Noe J M, Arndt K A, Rosen S. Port-wine stains. Morphologic variations and developmental lesions. Archives of Dermatology. 1984; 120:1453-1455.
[9]. Barsky S H, Rosen S, Geer D E, Noe J M. The nature and evolution of port wine stains: a computer-assisted study. Journal of Investigative Dermatology. 1980; 74:154-157.
[10]. Wagner K D, Wagner R F, Jr. The necessity for treatment of childhood port-wine stains. Cutis. 1990; 45:317-318.
[11]. Troilius A, Wrangsjo B, Ljunggren B. Patients with port-wine stains and their psychosocial reactions after photothermolytic treatment. Dermatologic Surgery. 2000; 26:190-196.
[12]. del Pozo J, Fonseca E. Port-wine stain nodules in the adult: report of 20 cases treated by CO2 laser vaporization. Dermatologic Surgery. 2001; 27:699-702.
[13]. Waner M, Suen J Y. Management of congenital vascular lesions of the head and neck. Oncology (Huntington). 1995; 9:989-994.
[14]. van der Horst C M, Koster P H, de Borgie C A, Bossuyt P M, van Gernert M J. Effect of the timing of treatment of port-wine stains with the flash-lamp-pumped pulsed-dye laser, [see comments]. New England Journal of Medicine. 1998; 338:1028-1033.
[15]. Waner M, Suen J Y. Hemangiomas and vascular malformations of the head and neck. New York: Wiley-Liss; 1999.
[16]. Shafirstein G, Buckmiller L M, Waner M, Baumler W. Mathematical modeling of selective photothermolysis to aid the treatment of vascular malformations and hemangioma with pulsed dye laser. Lasers Med Sci. 2007; 22:111-118.
[17]. Scherer K, Lorenz S, Wimmershoff M, Landthaler M, Hohenleutner U. Both the flashlamp-pumped dye laser and the long-pulsed tunable dye laser can improve results in port-wine stain therapy. Br J Dermatol, 2001; 145:79-84.
[18]. Huikeshoven M, Koster P H, de Borgie C A, Beek J F, van Gernert M J, van der Horst C M. Redarkening of port-wine stains 10 years after pulsed-dye-laser treatment. N Engl J. Med. 2007; 356:1235-1240.
[19]. Renfro L, Geronemus R G. Anatomical differences of port-wine stains in response to treatment with the pulsed dye laser. Arch Dermatol. 1993; 129:182-188.
[20]. Shafirstein G, Baumler W, Lapidoth M, Ferguson S, North P E, Waner M. A new mathematical approach to the diffusion approximation theory for selective photothermolysis modeling and its implication in laser treatment of port-wine stains. Lasers Surg Med. 2004; 34:335-347.
[21]. Baumler W, Vural E, Landthaler M, Muzzi F, Shafirstein G. The effects of intense pulsed light (IPL) on blood vessels investigated by mathematical modeling. Lasers Surg Med. 2007; 39:132-139.
[22]. Baumler W, Vogl A, Landthaler M, Waner M, Shafirstein G. Port wine stain laser therapy and the computer-assisted modeling of vessel coagulation using the finite elements method. Medical Laser Application. 2005:247-254.
[23]. Smoller B R, Rosen S. Port-wine stains. A disease of altered neural modulation of blood vessels? Archives of Dermatology. 1986; 122:177-179.
[24]. Fiskerstrand E J, Svaasand L O, Kopstad G, Ryggen K, Aase S. Photothermally induced vessel-wall necrosis after pulsed dye laser treatment: lack of response in port-wine stains with small sized or deeply located vessels. Journal of Investigative Dermatology. 1996; 107:671-675.
[25]. Pfefer T J, Smithies D J, Milner T E, van Gernert M J, Nelson J S, Welch A J. Bioheat transfer analysis of cryogen spray cooling during laser treatment of port wine stains. [erratum appears in Lasers Surg Med 2000; 26(5):following 495]. Lasers in Surgery & Medicine. 2000; 26:145-157.
[26]. Troilius A, Svendsen G, Ljunggren B. Ultrasound investigation of port wine stains. Acta Derm Venereol. 2000; 80:196-199.
[27]. Anderson R R, Parrish J A. Selective photothermolysis: precise microsurgery by selective absorption of pulsed radiation. Science. 1983; 220:524-527.
[28]. Anderson R R, Parrish J A. Microvasculature can be selectively damaged using dye lasers: a basic theory and experimental evidence in human skin. Lasers Surg Med. 1981; 1:263-276.
[29]. Welch A J, Gernert M J Cv. Optical-thermal response of laser-irradiated tissue. New York: Plenum Press; 1995.
[30]. van Gernert M J, Welch A J, Pickering J W, Tan O T. Laser treatment of port wine stains. In: Welch A J, van Gernert M J, eds. Optical-Thermal Response of Laser-irradiated Tissue. New York: Plenum Press; 1995:789-829.
[31]. Landthaler M, Hohenleutner U. Laser treatment of congenital vascular malformations. Int Angiol. 1990; 9:208-213.
[32]. Dierickx C C, Casparian J M, Venugopalan V, Farinelli W A, Anderson R R. Thermal relaxation of port-wine stain vessels probed in vivo: the need for 1-10-millisecond laser pulse treatment. Journal of Investigative Dermatology. 1995; 105:709-714.
[33]. Tanghetti E, Sierra R A, Sherr E A, Mirkov M. Evaluation of pulse-duration on purpuric threshold using extended pulse pulsed dye laser (cynosure V-star). Lasers Surg Med. 2002; 31:363-366.
[34]. Svaasand L O, Aguilar G, Viator J A, Randeberg L L, Kimel S, Nelson J S. Increase of dermal blood volume fraction reduces the threshold for laser-induced purpura: implications for port wine stain laser treatment. Lasers Surg Med. 2004; 34:182-188.
[35]. Garden J M, Tan O T, Kerschmann R, et al. Effect of dye laser pulse duration on selective cutaneous vascular injury. J Invest Dermatol. 1986; 87:653-657.
[36]. Aguilar G, Diaz S H, Layernia E J, Nelson J S. Cryogen spray cooling efficiency: improvement of port wine stain laser therapy through multiple-intermittent cryogen spurts and laser pulses. Lasers Surg Med. 2002; 31:27-35.
[37]. van Gernert M J, Welch A J, Pickering J W, Tan O T, Gijsbers G H. Wavelengths for laser treatment of port wine stains and telangiectasia. Lasers in Surgery & Medicine. 1995; 16:147-155.
[38]. Gabay S, Lucassen G W, Verkruysse W, van Gernert M J. Modelling the assessment of port wine stain parameters from skin surface temperature following a diagnostic laser pulse. Lasers Surg Med. 1997; 20:179-187.
[39]. van Gernert M J, Smithies D J, Verkruysse W, Milner T E, Nelson J S. Wavelengths for port wine stain laser treatment: influence of vessel radius and skin anatomy. Phys Med Biol. 1997; 42:41-50.
[40]. Lucassen G W, Verkruysse W, Keijzer M, van Gernert M J. Light distributions in a port wine stain model containing multiple cylindrical and curved blood vessels. Lasers Surg Med. 1996; 18:345-357.
[41]. Zhang R, Verkruysse W, Aguilar G, Nelson J S. Comparison of diffusion approximation and Monte Carlo based finite element models for simulating thermal responses to laser irradiation in discrete vessels. Phys Med Biol. 2005; 50:4075-4086.
[42]. Babilas P, Shafirstein G, Baumler W, et al. Selective photothermolysis of blood vessels following flashlamp-pumped pulsed dye laser irradiation: in vivo results and mathematical modelling are in agreement. J Invest Dermatol. 2005; 125:343-352.
[43]. Babilas P, Shafirstein G, Baler J, et al. Photothermolysis of blood vessels using indocyanine green and pulsed diode laser irradiation in the dorsal skinfold chamber model. Lasers Surg Med. 2007; 39:341-352.
[44]. Baumler W, Ulrich H, Hartl A, Landthaler M, Shafirstein G. Optimal parameters for the treatment of leg veins using Nd:YAG lasers at 1064 nm. Br J. Dermatol. 2006; 155:364-371.
[45]. Simon R. Optimal Two-Stage Designs for Phase II Clinical Trials', Controlled Clinical Trials. Vol. 10; 1989:1-10.

What is claimed is:

1. A method of personalized interactive laser treatment comprising:
(a) exposing a treatment site to low level radiation;
(b) measuring the temperature increase and thermal relaxation at said treatment site,
(c) determining in real time the vessel size distribution at said treatment site by means of a set of executable instructions residing in a computer readable storage medium on a computer; and
(d) applying a therapeutically effective amount of radiant exposure to said treatment site.

2. The method of claim 1, wherein said low level radiation of step (a) is provided by a low power laser.

3. The method of claim 1, wherein said therapeutically effective amount of radiation exposure of step (d) is supplied by a treatment laser.

4. The method of claim 3, wherein said low power laser is selected from the group consisting of PDL laser and NIR lasers.

5. The method of claim 4, wherein said low level radiation is applied at an energy density of 4-5 J/cm² for PDL lasers and 20 J/cm² for NIR lasers.

6. The method of claim 1, wherein said low level radiation of step (a) and said therapeutically effective amount of radiant exposure of step (d) are supplied by a single laser.

7. The method of claim 1, wherein said low level radiation of step (a) and said therapeutically effective amount of radiant exposure of step (d) are supplied by two different lasers.

8. The method of claim 1, wherein said temperature increase and said thermal relaxation is communicated from said infrared sensor to said computer.

9. The method of claim 1, wherein said vessel size distribution is calculated based on said temperature increase and said thermal relaxation using a mathematical algorithm implemented in said set of executable instructions residing in said computer readable storage medium on said computer.

10. The method of claim 1, wherein said vessel size distribution is retrieved based on said temperature increase and said thermal relaxation from a look-up table residing in a computer readable storage medium on said computer.

11. The method of claim 1, wherein the appropriate laser parameters are communicated from said computer to said laser based on said vessel size distribution.

12. The method of claim 1, wherein steps (a) through (d) are repeated in a cycle of less than about 10 seconds.

13. The method of claim 1, wherein said measuring of step (b) is provided by an infrared sensor.

14. The method of claim 1, wherein said measuring of step (b) is provided by an thermal camera.

15. An apparatus for personalized interactive laser treatment comprising:
a laser hand piece comprising an infrared sensor capable of measuring the temperature increase and thermal relaxation at a treatment site after exposure to low level radiation by said laser hand piece; and
a computer having a set of executable instructions residing on a computer readable storage medium in said computer for determining vessel size distribution in real time based on said temperature increase and thermal relaxation.

16. The apparatus of claim 15, wherein said laser hand piece is capable of communicating said temperature increase and said thermal relaxation to said computer.

17. The apparatus of claim 15, wherein said vessel size distribution is calculated based on said temperature increase and said thermal relaxation using a mathematical algorithm implemented in said set of executable instructions residing in said computer readable storage medium on said computer.

18. The apparatus of claim 15, wherein said executable instructions comprise a look-up table for retrieving said vessel size distribution based on said temperature increase and said thermal relaxation.

19. The apparatus of claim 15, wherein said computer communicates to said laser hand piece a therapeutically effective amount of radiation to expose to said treatment site based on said vessel size distribution.

20. The apparatus of claim 19, wherein said laser hand piece is capable of applying said therapeutically effective amount of radiation to said treatment site.

21. The apparatus of claim 15, wherein the optimal distance of radiation exposure is 3-5 cm from the skin surface.

22. The apparatus of claim 15, further comprising a physical spacer.

23. The apparatus of claim 15, further comprising a optical diffuser or filter.

24. The apparatus of claim 15, further comprising a low power laser port.

25. The apparatus of claim 15, wherein said infrared sensor is operatively coupled to one or more fiber optics.
